(19) United States
(12) Patent Application Publication
(10) Pub. No.: US 2013/0158292 A1
(43) Pub. Date: Jun. 20, 2013

(54) METHOD FOR PRODUCING METHIONINE

(71) Applicant: SUMITOMO CHEMICAL COMPANY, LIMITED, Tokyo (JP)

(72) Inventors: Koji HAGIYA, Osaka-shi (JP); Kazuyasu TANI, Osaka-shi (JP)

(73) Assignee: SUMITOMO CHEMICAL COMPANY, LIMITED, Tokyo (JP)

(21) Appl. No.: 13/713,774

(22) Filed: Dec. 13, 2012

(30) Foreign Application Priority Data
Dec. 14, 2011 (JP) 2011-273052

(51) Int. Cl. C07C 319/20

(52) U.S. Cl. CPC C07C 319/20
USPC 562/559

(57) ABSTRACT

A novel method capable of producing methionine without using hydrogen cyanide as a raw material has been desired. The present invention relates to a method for producing methionine including Step A of oxidizing 4-methylthio-2-oxo-1-butanal in the presence of an alcohol, and Step B of reductively aminating 4-methylthio-2-oxo-butyrate obtained in Step A. It is preferable that Step A is carried out by reacting 4-methylthio-2-oxo-1-butanal, the alcohol and an oxidizing agent in the presence of a carbene catalyst.

### BACKGROUND OF THE INVENTION

1. Field of the Invention

The present application is filed, claiming the Paris Convention priority based on Japanese Patent Application No. 2011-273052 (filed on Dec. 14, 2011), and the entire content of which is incorporated herein by reference.

The present invention relates to a method for producing methionine.

2. Description of the Related Art

Methionine is an essential amino acid and an important compound which can also serve as a feed additive.

Kogyo Yuki Kagaku (Industrial Organic Chemistry), Tokyo Kagaku Dojin, pages 273 to 275 (1978), for example, describes a method for producing methionine in which 3-methylthiopropionaldehyde, which is obtained by adding methanethiol to acrolein, is reacted with hydrogen cyanide to give 2-hydroxy-4-methylthiobutyronitrile, which is reacted with ammonium carbonate to give substituted hydantoin, and the substituted hydantoin is hydrolyzed with an alkali.

According to the method described in Kogyo Yuki Kagaku (Industrial Organic Chemistry), Tokyo Kagaku Dojin, pages 273 to 275 (1978), however, hydrogen cyanide, for which care should be exercised in handling, is used as a raw material, and sufficient management and facilities conforming thereto are necessary for handling hydrogen cyanide.

Under these circumstances, a novel method capable of producing methionine without using hydrogen cyanide as a raw material has been desired.

### SUMMARY OF THE INVENTION

The present inventors have made earnest study to solve the problem described above, and have reached the present invention.

That is, the present invention is as follows.

[1] A method for producing methionine comprising:

Step A of oxidizing 4-methylthio-2-oxo-1-butanal in the presence of an alcohol; and

Step B of reductively aminating 4-methylthio-2-oxo-butyrate obtained in Step A.

[2] The production method according to [1], wherein Step A is carried out by reacting 4-methylthio-2-oxo-1-butanal, the alcohol and an oxidizing agent in the presence of a carbene catalyst.
[3] The production method according to [2], wherein the carbene catalyst in Step A is at least one compound selected from the group consisting of a compound obtained by reacting a compound represented by the formula (2-1):

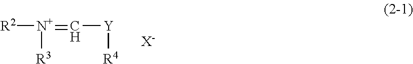

wherein R² is an optionally substituted alkyl group or an optionally substituted aryl group, R³ and R⁴ are each independently an optionally substituted alkyl group or an optionally substituted aryl group, or R³ and R⁴ are combined together to form an optionally substituted bivalent hydrocarbon group or an optionally substituted group represented by —CH═N—, Y is a group represented by —S— or a group represented by —N(R⁵)—, R⁵ is an optionally substituted alkyl group or an optionally substituted aryl group, or R⁵ and R⁴ are combined together to form an optionally substituted bivalent hydrocarbon group, and X⁻ is an anion, with a base; a compound represented by the formula (2-2):

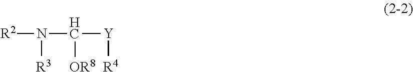

wherein R², R³, R⁴ and Y are each as defined above, and R⁸ is an alkyl group; a compound obtained by decomposing the compound represented by the formula (2-2); a compound represented by the formula (2-3):

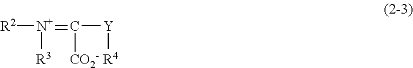

wherein R², R³, R⁴ and Y are each as defined above; and a compound obtained by decomposing the compound represented by the formula (2-3).

[4] The production method according to [2] or [3], wherein the oxidizing agent in Step A is oxygen and/or carbon dioxide.
[5] The production method according to any one of [1] to [4], wherein the alcohol is methanol or ethanol.
[6] The production method according to any one of [1] to [5], wherein Step B is carried out in the presence of a solvent.
[7] The production method according to [6], wherein the solvent in Step B is methanol or water.
[8] The production method according to any one of [1] to [7], wherein Step B is carried out by reacting 4-methylthio-2-oxo-butyrate, ammonia and a reducing agent in the presence of a transition metal.
[9] The production method according to [8], wherein the transition metal in Step B is at least one metal selected from the group consisting of ruthenium, rhodium, palladium, platinum, iridium, nickel, cobalt and copper.

According to the present invention, a novel method capable of producing methionine without using hydrogen cyanide as a raw material can be provided.

### DETAILED DESCRIPTION OF THE INVENTION

The present invention will be explained in detail below.

The method for producing methionine according to the present invention is characterized by comprising Step A of oxidizing 4-methylthio-2-oxo-1-butanal in the presence of an alcohol; and Step B of reductively aminating 4-methylthio-2-oxo-butyrate obtained in Step A. By carrying out the oxidation reaction of Step A and the reductive amination reaction of Step B, methionine can be produced without using hydrogen cyanide as a raw material.

First, Step A of oxidizing 4-methylthio-2-oxo-1-butanal in the presence of an alcohol will be explained. By carrying out Step A, 4-methylthio-2-oxo-butyrate can be obtained.

In Step A, 4-methylthio-2-oxo-1-butanal is oxidized in the presence of an alcohol, and preferably, 4-methylthio-2-oxo-1-butanal, an alcohol and an oxidizing agent are reacted in the presence of a carbene catalyst. Hereinafter, the reaction in Step A may be referred to as an “oxidation reaction.”

Preferable examples of the carbene catalyst used in Step A are at least one compound selected from the group consisting of a compound obtained by reacting a compound represented by the formula (2-1) (hereinafter sometimes referred to as “Compound (2-1)”) with a base; a compound represented by the formula (2-2) (hereinafter sometimes referred to as “Compound (2-2)”), a compound obtained by decomposing the compound represented by the formula (2-2); a compound represented by the formula (2-3) (hereinafter sometimes referred to as “Compound (2-3)”), and a compound obtained by decomposing the compound represented by the formula (2-3).

In the formula (2-1), examples of the alkyl group in the optionally substituted alkyl group represented by R³ and the optionally substituted alkyl group represented by R⁴ include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and cyclic C₃-C₁₂ alkyl groups such as a cyclopropyl group, a 2,2-dimethylcyclopropyl group, a cyclopentyl group, a cyclohexyl group and a menthyl group.

Examples of a substituent optionally possessed by the alkyl group in R³ and R⁴ include groups selected from Group G3 described below.

### <Group G3>

C₆-C₁₀ aryl groups optionally having a C₁-C₁₀ alkoxy group,

C₁-C₁₀ alkoxy groups optionally having a fluorine atom,

benzyloxy groups optionally having at least one kind of group selected from the group consisting of C₁-C₁₀ alkoxy groups, C₁-C₁₀ alkyl groups and C₆-C₁₀ aryloxy groups, C₆-C₁₀ aryloxy groups optionally having a C₁-C₁₀ alkoxy group,

C₆-C₁₀ aryloxy groups having a C₆-C₁₀ aryloxy group,

C₂-C₁₀ acyl groups optionally having a C₁-C₁₀ alkoxy group,

a carboxy group, and

a fluorine atom.

In Group G3, examples of the C₆-C₁₀ aryl groups optionally having a C₁-C₁₀ alkoxy group include a phenyl group, a naphthyl group, a 4-methylphenyl group and a 4-methoxyphenyl group;

examples of the C₁-C₁₀ alkoxy groups optionally having a fluorine atom include a methoxy group, an ethoxy group, a propoxy group, an isopropoxy group, a butoxy group, an isobutoxy group, a sec-butoxy group, a tert-butoxy group and a trifluoromethoxy group;

examples of the benzyloxy groups optionally having at least one kind of group selected from the group consisting of C₁-C₁₀ alkoxy groups, C₁-C₁₀ alkyl groups and C₆-C₁₀ aryloxy groups include a benzyloxy group, a 4-methylbenzyloxy group, a 4-methoxybenzyloxy group and a 3-phenoxybenzyloxy group;

examples of the C₆-C₁₀ aryloxy groups optionally having a C₁-C₁₀ alkoxy group include a phenoxy group, a 2-methylphenoxy group, a 4-methylphenoxy group and a 4-methoxyphenoxy group;

examples of the C₆-C₁₀ aryloxy groups having a C₆-C₁₀ aryloxy group include a 3-phenoxyphenoxy group; and

examples of the C₂-C₁₀ acyl groups optionally having a C₁-C₁₀ alkoxy group include an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group and a 4-methoxybenzoyl group.

Examples of the alkyl group having a group selected from Group G3 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group and a 2-carboxyethyl group.

In the formula (2-1), examples of the aryl group in the optionally substituted aryl group represented by R³ and the optionally substituted aryl group represented by R⁴ include C₆-C₁₀ aryl groups such as a phenyl group, a 2-methylphenyl group, a 4-methylphenyl group and a naphthyl group.

Examples of a substituent optionally possessed by the aryl group include the groups selected from Group G3 described above.

Examples of the aryl group having a group selected from Group G3 include a 4-chlorophenyl group and a 4-methoxyphenyl group.

In the formula (2-1), examples of the optionally substituted bivalent hydrocarbon group which is formed by combining R³ and R⁴ together include an ethylene group, a vinylene group, a propan-1,2-diyl group, a propen-1,2-diyl group, a butan-1,2-diyl group, a 2-buten-1,2-diyl group, a cyclopentan-1,2-diyl group, a cyclohexan-1,2-diyl group, an o-phenylene group, a 1,2-diphenylethylene group and a 1,2-diphenylvinylene group. Examples of the substituent by which the bivalent hydrocarbon group may be substituted include the groups selected from Group G3 described above. In the formula (2-1), examples of the substituent optionally possessed by the group represented by —CH═N—, which is formed by combining R³ and R⁴ together, include alkyl groups optionally having a group selected from Group G3 described above, and aryl groups optionally having a group selected from Group G3 described above. Examples of the alkyl group in the alkyl group optionally having a group selected from Group G3 include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and cyclic C₃-C₁₂ alkyl groups such as a cyclopropyl group, a 2,2-dimethylcyclopropyl group, a cyclopentyl group, a cyclohexyl group and a menthyl group. Examples of the aryl group in the aryl group optionally having a group selected from Group G3 include C₆-C₁₀ aryl groups such as a phenyl group, a 2-methylphenyl group, a 4-methylphenyl group and a naphthyl group.

With respect to R³ and R⁴, it is preferable that R³ and R⁴ are combined together to form the optionally substituted bivalent hydrocarbon group.

In the formula (2-1), examples of the alkyl group in the optionally substituted alkyl group represented by R² and the optionally substituted alkyl group represented by R⁵ include linear or branched C₁-C₁₂ alkyl groups such as a methyl group, an ethyl group, a propyl group, an isopropyl group, a butyl group, an isobutyl group, a sec-butyl group, a tert-butyl group, a pentyl group, a tert-pentyl group and a decyl group; and cyclic C₃-C₁₂ alkyl groups such as a cyclopropyl group, a 2,2-dimethylcyclopropyl group, a cyclopentyl group, a cyclohexyl group, a menthyl group and an adamantyl group.

Examples of a substituent optionally possessed by the alkyl group include groups selected from Group G4 described below.

### <Group G4>

C₆-C₁₀ aryl groups optionally having a C₁-C₁₀ alkoxy group,

C₁-C₁₀ alkoxy groups optionally having a fluorine atom,

C₇-C₂₀ aralkyloxy groups optionally having a C₁-C₁₀ alkoxy group,

C₇-C₂₀ aralkyloxy groups having a C₆-C₁₀ aryloxy group, C₆-C₁₀ aryloxy groups optionally having a C₁-C₁₀ alkoxy group,

C₆-C₁₀ aryloxy groups having a C₆-C₁₀ aryloxy group, and C₂-C₁₀ acyl groups optionally having a C₁-C₁₀ alkoxy group.

In Group G4, examples of the C₆-C₁₀ aryl groups optionally having a C₁-C₁₀ alkoxy group include a phenyl group, a naphthyl group, a 4-methylphenyl group and a 4-methoxyphenyl group;

examples of the C₁-C₁₀ alkoxy groups optionally having a fluorine atom include a methoxy group, an ethoxy group, a propoxy group, an isopropoxy group, a butoxy group, an isobutoxy group, a sec-butoxy group, a tert-butoxy group and a trifluoromethoxy group;

examples of the C₇-C₂₀ aralkyloxy groups optionally having a C₁-C₁₀ alkoxy group include a benzyloxy group, a 4-methylbenzyloxy group and a 4-methoxybenzyloxy group;

examples of the C₇-C₂₀ aralkyloxy groups having a C₆-C₁₀ aryloxy group include a 3-phenoxybenzyloxy group;

examples of the C₆-C₁₀ aryloxy groups optionally having a C₁-C₁₀ alkoxy group include a phenoxy group, a 2-methylphenoxy group, a 4-methylphenoxy group and a 4-methoxyphenoxy group;

examples of the C₆-C₁₀ aryloxy groups having a C₆-C₁₀ aryloxy group include a 3-phenoxyphenoxy group; and

examples of the C₂-C₁₀ acyl groups optionally having a C₁-C₁₀ alkoxy group include an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group and a 4-methoxybenzoyl group.

Examples of the alkyl group having a group selected from Group G4 include a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group and a phenacyl group.

In the formula (2-1), examples of the aryl group in the optionally substituted aryl group represented by R² and the optionally substituted aryl group represented by R⁵ include C₆-C₂₀ aryl groups such as a phenyl group, a naphthyl group, a 2-methylphenyl group, a 4-methylphenyl group, a 2,6-dimethylphenyl group, a 2,4,6-trimethylphenyl group and a 2,6-diisopropylphenyl group.

Examples of a substituent optionally possessed by the aryl group include groups selected from Group G5 described below.

### <Group G5>

C₁-C₁₀ alkoxy groups optionally having a fluorine atom or a C₁-C₁₀ alkoxy group, and

a halogen atom.

In Group G5, examples of the C₁-C₁₀ alkoxy groups optionally having a fluorine atom or a C₁-C₁₀ alkoxy group include a methoxy group, an ethoxy group, a propoxy group, an isopropoxy group, a butoxy group, an isobutoxy group, a sec-butoxy group, a tert-butoxy group, a pentyloxy group, a cyclopentyloxy group, a fluoromethoxy group, a trifluoromethoxy group, a methoxymethoxy group, an ethoxymethoxy group and a methoxyethoxy group; and examples of the halogen atom include a fluorine atom and a chlorine atom.

Examples of the aryl group having a group selected from Group G5 include a 4-chlorophenyl group, a 4-methoxyphenyl group and a 2,6-dichlorophenyl group.

In the formula (2-1), R⁵ and R⁴ may be combined together to form an optionally substituted bivalent hydrocarbon group. Examples of the bivalent hydrocarbon group include an ethylene group, polymethylene groups such as a trimethylene group and a tetramethylene group, a vinylene group, a propan-1,2-diyl group, a propen-1,2-diyl group, a butan-1,2-diyl group, a 2-buten-1,2-diyl group, a cyclopentan-1,2-diyl group, a cyclohexan-1,2-diyl group and an o-phenylene group. Examples of the substituent optionally possessed by the bivalent hydrocarbon group include the groups selected from Group G3 described above.

In the formula (2-1), examples of the anion represented by X⁻ include halide ions such as a chloride ion, a bromide ion and an iodide ion; ions of alkanesulfonates optionally having a fluorine atom such as methanesulfonate and trifluoromethanesulfonate; ions of acetates optionally having a halogen atom such as trifluoroacetate and trichloroacetate ions; a nitrate ion; a perchlorate ion; ions of tetrahaloborates such as tetrafluoroborate and tetrachloroborate; ions of hexahalophosphates such as hexafluorophosphate; ions of hexahaloantimonates such as hexafluoroantimonate and hexachloroantimonate; ions of pentahalostannates such as pentafluorostannate and pentachlorostannate; and optionally substituted tetraarylborates such as tetraphenylborate, tetrakis(pentafluorophenyl)borate and tetrakis[3,5-bis(trifluoromethyl)phenyl]borate.

Compound (2-1) is preferably a compound represented by the formula (2-4) (hereinafter sometimes referred to as “Compound (2-4)”):

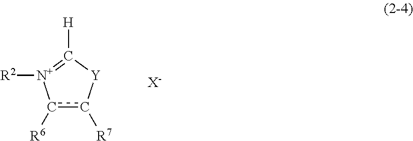

wherein R², Y and X⁻ are each as defined above; R⁶ and R⁷ are each independently a hydrogen atom, an optionally substituted alkyl group or an optionally substituted aryl group, or R⁶ and R⁷ are combined together with a carbon atom to which they are attached to form a ring, or R⁶ is a hydrogen atom, an optionally substituted alkyl group or an optionally substituted aryl group, and R⁷ and R⁵ are combined together to form an optionally substituted bivalent hydrocarbon group; and

[symbol]

is a single bond or a double bond, or a compound represented by the formula (2-5) (hereinafter sometimes referred to as “Compound (2-5)”):

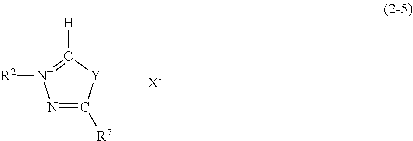

wherein R², Y and X⁻ are each as defined above; R⁷ is a hydrogen atom, an optionally substituted alkyl group or an optionally substituted aryl group, or R⁷ and R⁵ are combined together to form an optionally substituted bivalent hydrocarbon group. Compound (2-4) is more preferable.

Compound (2-4) and Compound (2-5) will be explained below.

In the formula (2-4) and the formula (2-5), R² has the same definition as R² in the formula (2-1), and Y has the same definition as Y in the formula (2-1). When Y is a group represented by —N(R⁵)— in the formula (2-4) and the formula (2-5), R⁵ has the same definition as R⁵ in the formula (2-1). X⁻ in the formula (2-4) and the formula (2-5) has the same definition as X⁻ in the formula (2-1).

In the formula (2-4), R² is preferably a bulky group. When Y is a group represented by —N(R⁵)—, it is preferable that either R² or R⁵ is a bulky group, and it is more preferable that both R² and R⁵ are bulky groups. R² and R⁵ may be the same group or different groups.

Examples of the bulky group in R² and R⁵ include C₄-C₁₂ tertiary alkyl groups such as a tert-butyl group and a tert-pentyl group; C₃-C₁₀ cycloalkyl groups such as a cyclopropyl group, a 2,2-dimethylcyclopropyl group, a cyclopentyl group, a cyclohexyl group, a menthyl group and an adamantyl group; phenyl groups having substituents at least at the 2-position and the 6-position (2,6-disubstituted phenyl groups) such as a 2,6-dimethylphenyl group, a 2,6-dichlorophenyl group, a 2,6-dibromophenyl group, a 2,4,6-trimethylphenyl group and a 2,6-diisopropylphenyl group; and naphthyl groups having a C₁-C₁₀ alkyl group at the 2-position such as a 2-methylnaphthyl group. Examples of the substituent in the 2,6-disubstituted phenyl group include C₁-C₁₂ alkyl groups and a halogen atom.

The bulky group in R² and R⁵ is preferably a tert-butyl group, a tert-pentyl group, a cyclohexyl group, an adamantyl group or a 2,6-disubstituted phenyl group, more preferably a 2,6-disubstituted phenyl group, and even more preferably a 2,6-dibromophenyl group or a 2,6-diisopropylphenyl group.

Examples of the alkyl group in the optionally substituted alkyl group represented by R⁶ in the formula (2-4) and the optionally substituted alkyl group represented by R⁷ in the formula (2-4) and the formula (2-5) include linear, branched or cyclic C₁-C₁₀ alkyl groups such as a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group and menthyl group.

Examples of a substituent optionally possessed by the alkyl group include groups selected from Group G3 described above. Examples of the alkyl group having a group selected from Group G3 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group and a 2-carboxyethyl group.

Examples of the aryl group in the optionally substituted aryl group represented by R⁶ in the formula (2-4) and the optionally substituted aryl group represented by R⁷ in the formula (2-4) and the formula (2-5) include C₆-C₁₀ aryl groups such as a phenyl group, a 2-methylphenyl group, a 4-methylphenyl group and a naphthyl group.

Examples of a substituent optionally possessed by the aryl group include groups selected from Group G3 described above.

Examples of the aryl group having a group selected from Group G3 include a 4-chlorophenyl group and a 4-methoxyphenyl group.

In the formula (2-4), R⁶ and R⁷ may be combined together with a carbon atom to which they are attached to form a ring. Examples of such a ring include a cyclopentane ring, a cyclohexane ring and a benzene ring.

In the formula (2-4), it is preferable that R⁶ and R⁷ are each independently a hydrogen atom or an optionally substituted alkyl group, and it is more preferable that both R⁶ and R⁷ are hydrogen atoms.

When Y is a group represented by —N(R⁵)— in the formula (2-4) and the formula (2-5), R⁵ and R⁷ may be combined together to form an optionally substituted bivalent hydrocarbon group. Examples of the bivalent hydrocarbon group include an ethylene group, polymethylene groups such as a trimethylene group and a tetramethylene group, a vinylene group, a propan-1,2-diyl group, a propen-1,2-diyl group, a butan-1,2-diyl group, a 2-buten-1,2-diyl group, a cyclopentan-1,2-diyl group, a cyclohexan-1,2-diyl group and an o-phenylene group. Examples of a substituent optionally possessed by the bivalent hydrocarbon group include groups selected from Group G3 described above.

In the formula (2-4), when Y is a group represented by —N(R⁵)—,

[symbol]

is preferably a single bond, and when Y is a group represented by —S—, [symbol]
is preferably a double bond.

Examples of Compound (2-4) include:

Compounds (2-4) wherein Y is a group represented by —N(R⁵)—; and R² and R⁵ are independently a C₄-C₁₂ tertiary alkyl group, a C₃-C₁₀ cycloalkyl group, a phenyl group having substituents at least at the 2-position and the 6-position, or a naphthyl group having a C₁-C₁₀ alkyl group at the 2-position;

Compounds (2-4) wherein Y is a group represented by —N(R⁵)—; R² and R⁵ are independently a C₄-C₁₂ tertiary alkyl group, a C₃-C₁₀ cycloalkyl group, a phenyl group having substituents at least at the 2-position and the 6-position, or a naphthyl group having a C₁-C₁₀ alkyl group at the 2-position; and

[symbol]

is a single bond;

Compounds (2-4) wherein Y is a group represented by —N(R⁵)—; and R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

Compounds (2-4) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position; and

[symbol]

is a single bond;

Compounds (2-4) wherein Y is a group represented by —N(R⁵)—; and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

Compounds (2-4) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and

[symbol]

is a single bond;

Compounds (2-4) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a single bond; and R⁶ and R⁷ are independently a hydrogen atom or a linear, branched or cyclic C₁-C₁₀ alkyl group optionally having a group selected from Group G3 described above;

Compounds (2-4) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a single bond; and R⁶ and R⁷ are hydrogen atoms;

Compounds (2-4) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

[symbol]

is a single bond; and R⁶ and R⁷ are hydrogen atoms;

Compounds (2-4) wherein Y is a group represented by —S—; and R² is a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position;

Compounds (2-4) wherein Y is a group represented by —S—; R² is a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position; and

[symbol]

is a double bond;

Compounds (2-4) wherein Y is a group represented by —S—; and R² is a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

Compounds (2-4) wherein Y is a group represented by —S—; R² is independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position; and

[symbol]

is a double bond;

Compounds (2-4) wherein Y is a group represented by —S—; and R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

Compounds (2-4) wherein Y is a group represented by —S—; R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and [symbol] is a double bond;

Compounds (2-4) wherein Y is a group represented by —S—; R² is independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a double bond; and R⁶ and R⁷ are independently a hydrogen atom or a linear, branched or cyclic C₁-C₁₀ alkyl group optionally having a group selected from Group G3 described above;

Compounds (2-4) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group or a menthyl group; and

Compounds (2-4) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; [symbol]
is a single bond; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group or a menthyl group.

Examples of Compound (2-4) include 1,3-di-tert-butylimidazolium chloride, 1,3-di-tert-butylimidazolinium chloride, 1,3-dicylohexylimidazolium chloride, 1,3-dicylohexylimidazolinium chloride, 1,3-diadamantylimidazolium chloride, 1,3-diphenylimidazolium chloride, 1,3-diphenylimidazolinium chloride, 1,3-bis[(2,6-diisopropyl)phenyl]imidazolium chloride, 1,3-bis[(2,6-diisopropyl)phenyl]imidazolinium chloride, 1,3-bis[(2,4,6-trimethyl)phenyl]imidazolium chloride, 1,3-bis[(2,4,6-trimethyl)phenyl]imidazolinium chloride, 1,3-bis[(2,6-dibromo)phenyl]imidazolinium chloride, 1,3-bis[(2,4,6-tribromo)phenyl]imidazolinium chloride, 4,5-dimethyl-1,3-bis[(2,4,6-trimethyl)phenyl]imidazolium chloride, 4,5-dimethyl-1,3-bis[(2,4,6-trimethyl)phenyl]imidazolinium chloride, 4,5-dimethyl-1,3-bis[(2,6-diisopropyl)phenyl]imidazolium chloride, 4,5-dimethyl-1,3-bis[(2,6-diisopropyl)phenyl]imidazolinium chloride, 4,5-dichloro-1,3-bis[(2,6-diisopropyl)phenyl]imidazolium chloride, 4,5-dichloro-1,3-bis[(2,6-diisopropyl)phenyl]imidazolinium chloride, 4,5-diphenyl-1,3-bis[(2,4,6-trimethyl)phenyl]imidazolium chloride, 4,5-diphenyl-1,3-bis[(2,4,6-trimethyl)phenyl]imidazolinium chloride, 4,5-difluoro-1,3-bis[(2,6-diisopropyl)phenyl]imidazolium chloride, 4,5-difluoro-1,3-bis[(2,6-diisopropyl)phenyl]imidazolinium chloride, 4-methyl-1,3-bis[(2,4,6-trimethyl)phenyl]imidazolium chloride, 4-methyl-1,3-bis[(2,4,6-trimethyl)phenyl]imidazolinium chloride, 1,3-bis[(2,6-dichloro)phenyl]imidazolium chloride, 1,3-bis[(2,6-dichloro)phenyl]imidazolinium chloride, 1-tert-butyl-3-phenylimidazolium chloride, 1-tert-butyl-3-phenylimidazolinium chloride, 1-cyclohexyl-3-[(2,6-diisopropyl)phenyl]imidazolium chloride, 1-cyclohexyl-3-[(2,6-diisopropyl)phenyl]imidazolinium chloride, 1-phenyl-3-[(2,4,6-trimethyl)phenyl]imidazolium chloride, 1-phenyl-3-[(2,4,6-trimethyl)phenyl]imidazolinium chloride, 1-tert-butyl-3-[(2,6-diisopropyl)phenyl]imidazolium chloride, 1-tert-butyl-3-[(2,6-diisopropyl)phenyl]imidazolinium chloride, 1-tert-butyl-3-[(2,4,6-trimethyl)phenyl]imidazolium chloride, 1-tert-butyl-3-[(2,4,6-trimethyl)phenyl]imidazolinium chloride, 3-ethylbenzothiazolium bromide, 3-butylbenzothiazolium chloride, 3-(2,6-diisopropyl)phenyl-4,5-dimethylthiazolium chloride, 3-phenyl-4,5-dimethylthiazolium chloride, 3-benzylthiazolium chloride, 3-benzyl-4-methylthiazolium chloride, 3-n-butyl-4-methylthiazolium chloride, 3-n-hexyl-4-methylthiazolium chloride, 3-cyclohexyl-4-methylthiazolium chloride, 3-n-octyl-4-methylthiazolium chloride and 3-(2,4,6-trimethyl)phenyl-4,5-dimethylthiazolium chloride.

Examples of Compound (2-5) include:

Compounds (2-5) wherein Y is a group represented by —N(R⁵)—; R² is a linear or branched C₁-C₁₂ alkyl group or a C₆-C₂₀ aryl group; and R⁵ is a linear or branched C₁-C₁₂ alkyl group or a C₆-C₂₀ aryl group;

Compounds (2-5) wherein Y is a group represented by —N(R⁵)—; R² is a linear or branched C₁-C₁₂ alkyl group or a C₆-C₂₀ aryl group; R⁵ is a linear or branched C₁-C₁₂ alkyl group or a C₆-C₂₀ aryl group; and R⁷ is a C₁-C₁₀ alkyl group or a C₆-C₁₀ aryl group;

Compounds (2-5) wherein Y is a group represented by —S—; and R² is a linear or branched C₁-C₁₂ alkyl group or a C₆-C₂₀ aryl group; and

Compounds (2-5) wherein Y is a group represented by —S—; R² is a linear or branched C₁-C₁₂ alkyl group or a C₆-C₂₀ aryl group; and R⁷ is a C₁-C₁₀ alkyl group or a C₆-C₁₀ aryl group.

Examples of Compound (2-5) include 1,4-dimethyl-1H-1,2,4-triazoli-4-umchloride, 1,3,4-triphenyl-1H-1,2,4-triazoli-4-umchloride, 3,5-diphenyl-1,3,4-thiadiazolium chloride, 3-methyl-5-phenyl-1,3,4-thiadiazolium chloride and 6,7-dihydro-2-pentafluorophenyl-5H-pyrrolo[2,1-c]-1,2,4-triazolium chloride.

In addition, examples of Compound (2-4) and Compound (2-5) also include Compounds (2-4) and Compounds (2-5) described above wherein the “chloride” is replaced by, for example, “iodide”, “bromide”, “methanesulfonate”, “trifluoromethanesulfonate”, “nitrate”, “perchlorate”, “tetrafluoroborate”, “tetrachloroborate”, “hexafluorophosphate”, “hexafluoroantimonate”, “hexachloroantimonate”, “pentafluorostannate”, “pentachlorostannate”, “tetraphenylborate”, “tetrakis(pentafluorophenyl)borate” or “tetrakis[3,5-bis(trifluoromethyl)phenyl]borate”.

Compound (2-1) may be a commercially available product, or can be produced according to a method described in, for example, J. Organometallic. Chem. Soc., 606, 49 (2000) or J. Org. Chem. Soc., 73, 2784 (2008).

The base, which is reacted with Compound (2-1) in Step A, is preferably at least one base selected from the group consisting of organic bases and alkali metal alkoxides. Examples of the organic base include tertiary amines such as triethylamine, trioctylamine, diisopropylethylamine and 4-dimethyl aminopyridine; nitrogen-containing aliphatic cyclic compounds such as 1,8-diazabicyclo[5,4,0]-7-undecene and 1,5,7-triazabicyclo[4,4,0]-5-decene; and nitrogen-containing aromatic compounds such as pyridine and imidazole.

Examples of the alkali metal in the alkali metal alkoxide include lithium, sodium and potassium. Examples of the alkoxide include methoxide, ethoxide, n-propoxide, isopropoxide, t-butoxide and sec-butoxide. At least one alkali metal alkoxide selected from the group consisting of lithium alkoxide, sodium alkoxide and potassium alkoxide is preferable.

The alkali metal alkoxide may be a high purity product, or may be an alcohol solution. In this case, an alcohol solvent contained in the alcohol solution is preferably the same alcohol as that used in Step A, because 4-methylthio-2-oxo-butyrate can be obtained in a high purity.

The amount of the base used for the reaction with Compound (2-1) in Step A is, for example, within a range of 0.1 mole to 10 moles, preferably within a range of 0.5 mole to 3 moles per mole of Compound (2-1).

A method for generating a carbene catalyst by reacting Compound (2-1) with the base will be described below.

The carbene catalyst may be generated at the same time with the oxidation reaction of Step A, as described below, or the carbene catalyst may be previously generated and then added to the oxidation reaction system of Step A.

When the carbene catalyst is generated at the same time with the oxidation reaction of Step A, a solvent may or may not be used. When the carbene catalyst is previously generated, it is preferable to generate the carbene catalyst in the presence of a solvent. Solvents which do not react with the generated carbene catalyst are preferably used, and examples of the solvent include ether solvents such as tetrahydrofuran, methyl tert-butyl ether, cyclopentyl methyl ether and diisopropyl ether; ester solvents such as ethyl acetate and butyl acetate; aromatic solvents such as toluene and chlorobenzene; nitrile solvents such as acetonitrile and propionitrile; and mixed solvents thereof.

The amount of the solvent used is not limited. For example, it is practically 100 parts by weight or less based on 1 part by weight of Compound (2-1).

In the generation of the carbene catalyst, the mixing order of reaction reagents is not limited. A preferable embodiment is an embodiment in which Compound (2-1) and the solvent are mixed, and a base is added to the resulting mixture.

The carbene catalyst can be generated in any condition of a reduced pressure, an ordinary pressure and an increased pressure, and the carbene catalyst is generated preferably in an ordinary pressure or an increased pressure.

The reaction temperature at which the carbene catalyst is generated varies depending on the kind of Compound (2-1), the kind of the base, the amount of use, the kind of the carbene catalyst generated or the like, and it is preferably within a range of −20° C. to 100° C., more preferably within a range of 0° C. to 50° C. When the reaction temperature is lower than −20° C., the generation speed of the carbene catalyst tends to be lowered, and when the reaction temperature is higher than 100° C., the carbene catalyst generated tends to be decomposed.

The degree of progress of the reaction in the generation of the carbene catalyst can be confirmed by an analysis procedure such as thin-layer chromatography, a nuclear magnetic resonance spectroscopic analysis, or an infrared absorption spectroscopic analysis.

After the reaction of generating the carbene catalyst is finished, the reaction liquid containing the carbene catalyst can be used as it is in the oxidation reaction of Step A, or production of Compound (2-2) or Compound (2-3) described below. In addition, after salts formed by the reaction with the base used are removed, if necessary, through filtration, the obtained reaction mixture is, if necessary, subjected to a concentration treatment, and then a cooling treatment or the like is carried out, whereby the carbene catalyst can be taken out.

Next, Compound (2-2) and Compound (2-3) will be explained.

Compound (2-2) can be obtained by reacting a compound, which is obtained by reacting Compound (2-1) with a base, with an alcohol represented by R⁸OH. Compound (2-3) can be obtained by reacting a compound, which is obtained by reacting Compound (2-1) with a base, with carbon dioxide. Here, R², R³, R⁴ and Y in the formula (2-2) and the formula (2-3) each have the same definition as those in the formula (2-1).

In the formula (2-2), examples of the alkyl group represented by R⁸ include linear or branched C₁-C₆ alkyl groups such as a methyl group, an ethyl group, a propyl group, an isopropyl group, a butyl group, an isobutyl group, a sec-butyl group, a pentyl group and a hexyl group.

The compound represented by the formula (2-2) is preferably a compound represented by the formula (2-6):

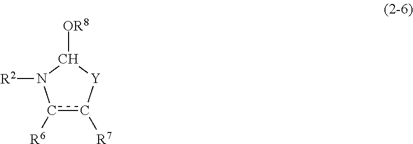

wherein R², R⁸, R⁶, R⁷ and Y are each as defined above, and

[symbol]

is a single bond or a double bond (hereinafter sometimes referred to as “Compound (2-6)”) or a compound represented by the formula (2-7):

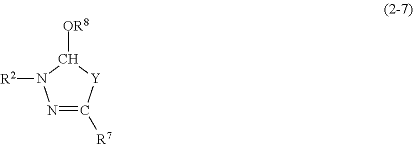

wherein R², R⁷, R⁸ and Y are each as defined above (hereinafter sometimes referred to as “Compound (2-7)”). Compound (2-6) is more preferable.

The compound represented by the formula (2-3) is preferably a compound represented by the formula (2-8):

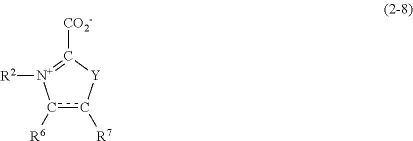

wherein R², R⁶, R⁷, Y and

[symbol]

are each as defined above (hereinafter sometimes referred to as “Compound (2-8)”), or a compound represented by the formula (2-9):

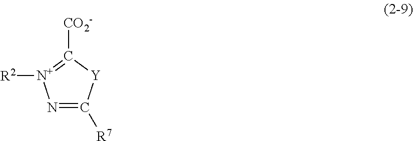

wherein R², R⁷ and Y are each as defined above (hereinafter sometimes referred to as “Compound (2-9)”). Compound (2-8) is more preferable.

Compound (2-6), Compound (2-7), Compound (2-8) and Compound (2-9) will be explained below.

In the formula (2-6), the formula (2-7), the formula (2-8) and the formula (2-9), R² has the same definition as R² in the formula (2-1); and Y has the same definition as Y in the formula (2-1). When Y is a group represented by —N(R⁵)—, R⁵ has the same definition as R⁵ in the formula (2-1). In the formula (2-6) and the formula (2-7), R⁸ has the same definition as R⁸ in the formula (2-2).

In the formula (2-6), the formula (2-7), the formula (2-8) and the formula (2-9), Y is preferably a group represented by —N(R⁵)—.

In the formula (2-6), the formula (2-7), the formula (2-8) and the formula (2-9), it is preferable that at least either R² or R⁵ is a bulky group, and it is more preferable that both R² and R⁵ are bulky groups. R² and R⁵ may be the same group or different groups.

Here, examples of the bulky group in R² and R⁵ include:

C₄-C₁₂ tertiary alkyl groups such as a tert-butyl group and a tert-pentyl group;

C₃-C₁₀ cycloalkyl groups such as a cyclopropyl group, a 2,2-dimethylcyclopropyl group, a cyclopentyl group, a cyclohexyl group, a menthyl group and an adamantyl group;

phenyl groups having substituents at least at the 2-position and the 6-position (2,6-disubstituted phenyl groups) such as a 2,6-dimethylphenyl group, a 2,6-dichlorophenyl group, a 2,4,6-trimethylphenyl group and a 2,6-diisopropylphenyl group; and

naphthyl groups which has a C₁-C₁₀ alkyl group at the 2-position such as a 2-methylnaphthyl group. Examples of the substituent in the 2,6-disubstituted phenyl group include a C₁-C₁₂ alkyl group and a halogen atom.

The bulky group is preferably a tert-butyl group, a tert-pentyl group, a cyclohexyl group, an adamantyl group or a 2,6-disubstituted phenyl group, more preferably a 2,6-disubstituted phenyl group, and even more preferably a 2,6-diisopropylphenyl group.

In the formula (2-6) and the formula (2-8), R⁶ has the same definition as R⁶ in the formula (2-4), and in the formula (2-6), the formula (2-7), the formula (2-8) and the formula (2-9), R⁷ has the same definition as R⁷ in the formula (2-5).

In the formula (2-6) and the formula (2-8), it is preferable that R⁶ and R⁷ are each independently a hydrogen atom or an optionally substituted alkyl group, and it is more preferable that both R⁶ and R⁷ are hydrogen atoms.

In the formula (2-6) and the formula (2-8),

[symbol]

is preferably a single bond.

Examples of Compound (2-6) include:

Compounds (2-6) wherein Y is a group represented by —N(R⁵)—; and R² and R⁵ are independently a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position;

Compounds (2-6) wherein Y is a group represented by —N(R⁵)—; R² and R⁵ are independently a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position; and

[symbol]

is a single bond;

Compounds (2-6) wherein Y is a group represented by —N(R⁵)—; and R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

Compounds (2-6) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position; and

[symbol]

is a single bond;

Compounds (2-6) wherein Y is a group represented by —N(R⁵)—; and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

Compounds (2-6) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and

[symbol]

is a single bond;

Compounds (2-6) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a single bond; and R⁶ and R⁷ are independently a hydrogen atom or a linear, branched or cyclic C₁-C₁₀ alkyl group optionally having a group selected from Group G3 described above;

Compounds (2-6) wherein Y is a group represented by —N(R⁵)—; R² and R⁵ are independently, a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a single bond; and R⁶ and R⁷ are hydrogen atoms; Compounds (2-6) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

Compounds (2-6) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

[symbol]

is a single bond; and R⁶ and R⁷ are hydrogen atoms;

Compounds (2-6) wherein Y is a group represented by —S—; R² is a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position;

Compounds (2-6) wherein Y is a group represented by —S—; R² is a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position; and

[symbol]

is a double bond;

Compounds (2-6) wherein Y is a group represented by —S—; R² is a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

Compounds (2-6) wherein Y is a group represented by —S—; R² is independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position; and

[symbol]

is a double bond;

Compounds (2-6) wherein Y is a group represented by —S—; and R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

Compounds (2-6) wherein Y is a group represented by —S—; R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and

[symbol]

is a double bond;

Compounds (2-6) wherein Y is a group represented by —S—; R² is independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a double bond; and R⁶ and R⁷ are independently a hydrogen atom or a linear, branched or cyclic C₁-C₁₀ alkyl group optionally having a group selected from Group G3 described above;

Compounds (2-6) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group or a menthyl group; and

Compounds (2-6) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

[symbol]

is a single bond; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group or a menthyl group.

Examples of Compound (2-6) include 2-methoxy-1,3-di-tert-butylimidazolidine, 2-ethoxy-1,3-di-tert-butylimidazolidine, 2-n-propoxy-1,3-di-tert-butylimidazolidine, 2-methoxy-1,3-dicyclohexylimidazolidine, 2-ethoxy-1,3-dicyclohexylimidazolidine, 2-propoxy-1,3-dicyclohexylimidazolidine, 2-methoxy-1,3-diadamantylimidazolidine, 2-methoxy-1,3-diphenylimidazolidine, 2-methoxy-1,3-bis[(2,6-diisopropyl)phenyl]imidazolidine, 2-methoxy-1,3-bis[(2,4,6-trimethyl)phenyl]imidazolidine, 2-ethoxy-1,3-bis[(2,6-diisopropyl)phenyl]imidazolidine, 2-ethoxy-1,3-bis[(2,4,6-trimethyl)phenyl]imidazolidine, 2-propoxy-1,3-bis[(2,6-diisopropyl)phenyl]imidazolidine, 2-propoxy-1,3-bis[(2,4,6-trimethyl)phenyl]imidazolidine, 2-butoxy-1,3-bis[(2,6-diisopropyl)phenyl]imidazolidine, 2-butoxy-1,3-bis[(2,4,6-trimethyl)phenyl]imidazolidine, 2-isopropoxy-1,3-bis[(2,6-diisopropyl)phenyl]imidazolidine, 2-isopropoxy-1,3-bis[(2,4,6-trimethyl)phenyl]imidazolidine, 2-methoxy-4,5-dimethyl-1,3-bis[(2,6-diisopropyl)phenyl]imidazolidine, 2-methoxy-4,5-dimethyl-1,3-bis[(2,4,6-trimethyl)phenyl]imidazolidine, 2-ethoxy-4,5-dimethyl-1,3-bis[(2,6-diisopropyl)phenyl]imidazolidine, 2-ethoxy-4,5-dimethyl-1,3-bis[(2,4,6-trimethyl)phenyl]imidazolidine, 2-methoxy-4,5-dichloro-1,3-bis[(2,6-diisopropyl)phenyl]imidazolidine, 2-methoxy-4,5-diphenyl-1,3-bis[(2,4,6-trimethyl)phenyl]imidazolidine, 2-methoxy-4,5-difluoro-1,3-bis[(2,6-diisopropyl)phenyl]imidazolidine,
2-methoxy-4-methyl-1,3-bis[(2,4,6-trimethyl)phenyl]imidazolidine, 2-methoxy-1,3-bis[(2,6-dichloro)phenyl]imidazolidine, 2-methoxy-1-tert-butyl-3-phenylimidazolidine, 2-methoxy-1-cyclohexyl-3-[(2,6-diisopropyl)phenyl]imidazolidine, 2-methoxy-1-phenyl-3-[(2,4,6-trimethyl)phenyl]imidazolidine, 2-ethoxy-1-tert-butyl-3-[(2,6-diisopropyl)phenyl]imidazolidine and 2-ethoxy-1-tert-butyl-3-[(2,4,6-trimethyl)phenyl]imidazolidine.

Examples of Compound (2-7) include:

Compounds (2-7) wherein Y is a group represented by —N(R⁵)—; R² is a linear or branched C₁-C₁₂ alkyl group or a C₆-C₂₀ aryl group; and R⁵ is a linear or branched C₁-C₁₂ alkyl group or a C₆-C₂₀ aryl group;

Compounds (2-7) wherein Y is a group represented by —N(R⁵)—; R² is a linear or branched C₁-C₁₂ alkyl group or a C₆-C₂₀ aryl group; R⁵ is a linear or branched C₁-C₁₂ alkyl group or a C₆-C₂₀ aryl group; and R⁷ is a C₁-C₁₀ alkyl group or a C₆-C₁₀ aryl group;

Compounds (2-7) wherein Y is a group represented by —S—; and R² is a linear or branched C₁-C₁₂ alkyl group or a C₆-C₂₀ aryl group; and

Compounds (2-7) wherein Y is a group represented by —S—; R² is a linear or branched C₁-C₁₂ alkyl group or a C₆-C₂₀ aryl group; and R⁷ is a C₁-C₁₀ alkyl group or a C₆-C₁₀ aryl group.

Examples of Compound (2-7) include 5-methoxy-1,4-dimethyl-1,2,4(5H)-triazoline and 5-methoxy-1,3,4-triphenyl-1,2,4(5H)-triazoline.

Examples of Compound (2-8) include:

Compounds (2-8) wherein Y is a group represented by —N(R⁵)—; R² and R⁵ are independently a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position;

Compounds (2-8) wherein Y is a group represented by —N(R⁵)—; R² and R⁵ are independently a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position; and

[symbol]

is a single bond;

Compounds (2-8) wherein Y is a group represented by —N(R⁵)—; and R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

Compounds (2-8) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position; and

[symbol]

is a single bond;

Compounds (2-8) wherein Y is a group represented by —N(R⁵)—; and R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

Compounds (2-8) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and

[symbol]

is a single bond;

Compounds (2-8) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a single bond; and R⁶ and R⁷ are independently a hydrogen atom or a linear, branched or cyclic C₁-C₁₀ alkyl group optionally having a group selected from Group G3 described above;

Compounds (2-8) wherein Y is a group represented by —N(R⁵)—; R² and R⁵ are independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a single bond; and R⁶ and R⁷ are hydrogen atoms;

Compounds (2-8) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

[symbol]

is a single bond; and R⁶ and R⁷ are hydrogen atoms;

Compounds (2-8) wherein Y is a group represented by —S—; and R² is a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position;

Compounds (2-8) wherein Y is a group represented by —S—; R² is a C₄-C₁₂ tertiary alkyl group, a C₃-C₁₀ cycloalkyl group, a phenyl group having substituents at least at the 2-position and the 6-position or a naphthyl group having a C₁-C₁₀ alkyl group at the 2-position; and

[symbol]

is a double bond;

Compounds (2-8) wherein Y is a group represented by —S—; and R² is a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

Compounds (2-8) wherein Y is a group represented by —S—; R² is independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position; and [symbol] is a double bond;

Compounds (2-8) wherein Y is a group represented by —S—; R² is a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

Compounds (2-8) wherein Y is a group represented by —S—; R² is a tert-butyl group, a tert-pentyl group, a cyclohexyl group, a adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and

[symbol]

is a double bond;

Compounds (2-8) wherein Y is a group represented by —S—; R² is independently a C₄-C₁₂ tertiary alkyl group, a C₃-C₁₀ cycloalkyl group, a phenyl group having C₁-C₁₂ alkyl groups or halogen atoms at least at the 2-position and the 6-position or a naphthyl group having a C₁-C₁₀ alkyl group at the 2-position;

[symbol]

is a double bond; and R⁶ and R⁷ are independently a hydrogen atom or a linear, branched or cyclic C₁-C₁₀ alkyl group optionally having a group selected from Group G3 described above;

Compounds (2-8) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group or a menthyl group; and

Compounds (2-8) wherein Y is a group represented by —N(R⁵)—; R² and R⁵ are independently a tert-butyl group, a tert-pentyl group, a cyclohexyl group, an adamantyl group, a 2,6-dimethylphenyl group, a 2,6-dichlorophenyl group, a 2,6-dibromophenyl group, a 2,4,6-trimethylphenyl group or a 2,6-diisopropylphenyl group;

[symbol]

is a single bond; and R⁶ and R⁷ are independently a hydrogen atom, a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group or a menthyl group.

Examples of Compound (2-8) include 2-carboxy-4,5-dihydro-1,3-di-tert-butylimidazolium, 2-carboxy-4,5-dihydro-1,3-dicyclohexylimidazolium, 2-carboxy-4,5-dihydro-1,3-diadamantylimidazolium, 2-carboxy-4,5-dihydro-1,3-diphenylimidazolium, 2-carboxy-4,5-dihydro-1,3-bis[(2,6-diisopropyl)phenyl]imidazolium, 2-carboxy-4,5-dihydro-1,3-bis[(2,4,6-trimethyl)phenyl]imidazolium, 2-carboxy-4,5-dihydro-4,5-dimethyl-1,3-bis[(2,6-diisopropyl)phenyl]imidazolium, 2-carboxy-4,5-dihydro-4,5-dimethyl-1,3-bis[(2,4,6-trimethyl)phenyl]imidazolium, 2-carboxy-4,5-dihydro-4,5-dichloro-1,3-bis[(2,6-diisopropyl)phenyl]imidazolium, 2-carboxy-4,5-dihydro-4,5-diphenyl-1,3-bis[(2,4,6-trimethyl)phenyl]imidazolium, 2-carboxy-4,5-dihydro-4,5-difluoro-1,3-bis[(2,6-diisopropyl)phenyl]imidazolium, 2-carboxy-4,5-dihydro-4-methyl-1,3-bis[(2,4,6-trimethyl)phenyl]imidazolium, 2-carboxy-4,5-dihydro-1,3-bis[(2,6-dichloro)phenyl]imidazolium, 2-carboxy-4,5-dihydro-1-tert-butyl-3-phenylimidazolium, 2-carboxy-4,5-dihydro-1-cyclohexyl-3-[(2,6-diisopropyl)phenyl]imidazolium, 2-carboxy-4,5-dihydro-1-phenyl-3-[(2,4,6-trimethyl)phenyl]imidazolium, 2-carboxy-4,5-dihydro-1-tert-butyl-3-[(2,6-diisopropyl)phenyl]imidazolium and 2-carboxy-4,5-dihydro-1-tert-butyl-3-[(2,4,6-trimethyl)phenyl]imidazolium.

Examples of Compound (2-9) include:

Compounds (2-9) wherein Y is a group represented by —N(R⁵)—; R² is a linear or branched C₁-C₁₂ alkyl group or a C₆-C₂₀ aryl group; and R⁵ is a linear or branched C₁-C₁₂ alkyl group or a C₆-C₂₀ aryl group; Compounds (2-9) wherein Y is a group represented by —N(R⁵)—; R² is a linear or branched C₁-C₁₂ alkyl group or a C₆-C₂₀ aryl group; R⁵ is a linear or branched C₁-C₁₂ alkyl group or a C₆-C₂₀ aryl group; and R⁷ is a C₁-C₁₀ alkyl group or a C₆-C₁₀ aryl group; Compounds (2-9) wherein Y is a group represented by —S—; and R² is a linear or branched C₁-C₁₂ alkyl group or a C₆-C₂₀ aryl group; and

Compounds (2-9) wherein Y is a group represented by —S—; R² is a linear or branched C₁-C₁₂ alkyl group or a C₆-C₂₀ aryl group; and R⁷ is a C₁-C₁₀ alkyl group or a C₆-C₁₀ aryl group.

Examples of Compound (2-9) include 5-carboxy-1,3,4-triphenyl-4H-1,2,4-triazolium.

Examples of Compound (2-2) and Compound (2-3) include commercially available products and those produced according to a method described in, for example, J. Am. Chem. Soc., Vol. 127, page 9079 (2005).

The method for generating the carbene catalyst by decomposition of Compound (2-2) will be described below.

Preferably, Compound (2-2) is heated to a predetermined temperature to decompose into a carbene catalyst and an alcohol, and the alcohol is removed therefrom, whereby the carbene catalyst can be generated.

The carbene catalyst may be generated at the same time with the oxidation reaction of Step A, as described below, or the carbene catalyst may be previously generated and then added to the oxidation reaction system of Step A.

When the carbene catalyst is generated at the same time with the oxidation reaction of Step A, a solvent may or may not be used. When the carbene catalyst is previously generated, it is preferable to generate the carbene catalyst in the presence of a solvent. Solvents which do not react with the generated carbene catalyst are preferably used, and examples of the solvent include ether solvents such as tetrahydrofuran, methyl tert-butyl ether, cyclopentyl methyl ether and diisopropyl ether; ester solvents such as ethyl acetate and butyl acetate; aromatic solvents such as toluene and chlorobenzene; nitrile solvents such as acetonitrile and propionitrile; and mixed solvents thereof.

The amount of the solvent used is not limited. For example, it is practically 100 parts by weight or less based on 1 part by weight of Compound (2-2).

In the generation of the carbene catalyst, the mixing order of reaction reagents is not limited. A preferable embodiment is an embodiment in which after Compound (2-2) and the solvent are mixed, the resulting mixture is heated to a predetermined temperature to distill an alcohol generated away.

The carbene catalyst can be generated in any condition of a reduced pressure, an ordinary pressure and an increased pressure, and the carbene catalyst is generated preferably in an ordinary pressure or a reduced pressure.

The reaction temperature at which the carbene catalyst is generated varies depending on the kind of Compound (2-2), the kind of the carbene catalyst generated or the like, and it is preferably within a range of −20° C. to 100° C., more preferably within a range of 0° C. to 50° C. When the reaction temperature is lower than −20° C., the generation speed of the carbene catalyst tends to be lowered, and when the reaction temperature is higher than 100° C., the carbene catalyst generated tends to be decomposed.

The degree of progress of the reaction in the generation of the carbene catalyst can be confirmed by an analysis procedure such as thin-layer chromatography, a nuclear magnetic resonance spectroscopic analysis, or an infrared absorption spectroscopic analysis.

After the reaction of generating the carbene catalyst is finished, the reaction liquid containing the carbene catalyst can be used as it is in the oxidation reaction of Step A. In addition, after the obtained reaction mixture is subjected to, for example, a concentration treatment, if necessary, a cooling treatment or the like is carried out, whereby the carbene catalyst can be taken out.

The method for generating the carbene catalyst by decomposition of Compound (2-3) will be explained below.

Preferably, Compound (2-3) is heated to a predetermined temperature to decompose into a carbene catalyst and carbon dioxide, and carbon dioxide is removed therefrom, whereby the carbene catalyst can be generated.

The carbene catalyst may be generated at the same time with the oxidation reaction of Step A, as described below, or the carbene catalyst may be previously generated and then added to the oxidation reaction system of Step A.

When the carbene catalyst is generated at the same time with the oxidation reaction of Step A, a solvent may or may not be used. When the carbene catalyst is previously generated, it is preferable to generate the carbene catalyst in the presence of a solvent. Solvents which do not react with the generated carbene catalyst are preferably used, and examples of the solvent include ether solvents such as tetrahydrofuran, methyl tert-butyl ether, cyclopentyl methyl ether and diisopropyl ether; ester solvents such as ethyl acetate and butyl acetate; aromatic solvents such as toluene and chlorobenzene; nitrile solvents such as acetonitrile and propionitrile; and mixed solvents thereof.

The amount of the solvent used is not limited. For example, it is practically 100 parts by weight or less based on 1 part by weight of Compound (2-3).

In the generation of the carbene catalyst, the mixing order of reaction reagents is not limited. A preferable embodiment is an embodiment in which after Compound (2-3) and the solvent are mixed, the resulting mixture is heated to a predetermined temperature to remove carbon dioxide.

The carbene catalyst can be generated in any condition of a reduced pressure, an ordinary pressure and an increased pressure, and the carbene catalyst is generated preferably in an ordinary pressure or a reduced pressure.

The reaction temperature at which the carbene catalyst is generated varies depending on the kind of Compound (2-3), the kind of the carbene catalyst generated or the like, and it is preferably within a range of −20° C. to 100° C., more preferably within a range of 0° C. to 50° C. When the reaction temperature is lower than −20° C., the generation speed of the carbene catalyst tends to be lowered, and when the reaction temperature is higher than 100° C., the carbene catalyst generated tends to be decomposed.

The degree of progress of the reaction in the generation of the carbene catalyst can be confirmed by an analysis procedure such as thin-layer chromatography, a nuclear magnetic resonance spectroscopic analysis, or an infrared absorption spectroscopic analysis.

After the reaction of generating the carbene catalyst is finished, the reaction liquid containing the carbene catalyst can be used as it is in the oxidation reaction of Step A. In addition, after the obtained reaction mixture is subjected to, for example, a concentration treatment, if necessary, a cooling treatment or the like is carried out, whereby the carbene catalyst can be taken out.

In Step A, it is preferable that 4-methylthio-2-oxo-1-butanal, the alcohol and the oxidizing agent are reacted in the presence of a carbene catalyst, as described above; it is more preferable that 4-methylthio-2-oxo-1-butanal, the alcohol and the oxidizing agent are reacted in the presence of at least one compound selected from the group consisting of the compound obtained by reacting the compound represented by the formula (2-1) with the base, the compound represented by the formula (2-2), the compound obtained by decomposing the compound represented by the formula (2-2), the compound represented by the formula (2-3), and the compound obtained by decomposing the compound represented by the formula (2-3); and it is even more preferable that 4-methylthio-2-oxo-1-butanal, the alcohol and the oxidizing agent are reacted in the presence of the compound obtained by reacting the compound represented by the formula (2-1) with the base.

The amount of the carbene catalyst used is preferably 0.001 mole to 0.5 mole, more preferably 0.01 mole to 0.3 mole per mole of 4-methylthio-2-oxo-1-butanal.

The alcohol used in Step A will be explained.

In Step A, the kind of the alcohol is not limited, and lower alcohols having 1 to 8 carbon atoms are preferably used. Examples thereof include methanol, ethanol, 1-propanol, isopropanol, 1-butanol, isobutanol, 1-pentanol, 1-hexanol, cyclohexanol and benzyl alcohol. Methanol and ethanol are more preferable.

Examples of the alcohol used in Step A include commercially available products and those produced according to any publicly known method. Examples of the publicly known method include a method of partially oxidizing alkane-substituted or alkyl-substituted benzene, a method of adding water to a double bond, and a method of producing an alcohol by a fermentation process.

In Step A, the amount of alcohol used is preferably 1 mole or more per mole of 4-methylthio-2-oxo-1-butanal, and the upper limit is not limited. However, the amount of alcohol is preferably 100 moles or less in terms of economy.

The oxidizing agent used in Step A is not particularly limited so long as it does not preferentially promote side reactions, and examples thereof include oxygen, carbon dioxide, manganese dioxide, azobenzene, quinone, benzoquinone and anthraquinone. A preferable oxidizing agent is at least one agent selected from the group consisting of oxygen and carbon dioxide.

Oxygen which can be used as the oxidizing agent in Step A may be an oxygen gas, an oxygen gas diluted with an inert gas such as nitrogen, or oxygen contained in the air. In addition, oxygen contained in the air, which is diluted with an inert gas such as nitrogen, may be used. The amount of oxygen used is preferably within a range of 1 to 100 moles per mole of 4-methylthio-2-oxo-1-butanal.

Carbon dioxide which can be used as the oxidizing agent in Step A may be in a gaseous state, a solid state (dry ice), or a supercritical state. Gaseous carbon dioxide may be diluted with an inert gas such as nitrogen.

The amount of carbon dioxide used is preferably 1 mole or more per mole of 4-methylthio-2-oxo-1-butanal, and the upper limit thereof is not limited. However, the amount is, for example, 100 moles or less in terms of the productivity.

Step A can be carried out in the presence of a solvent.

The solvent is not limited so long as it does not inhibit the production of 4-methylthio-2-oxo-butyrate in Step A, and examples thereof include ether solvents such as tetrahydrofuran, methyl tert-butyl ether, cyclopentyl methyl ether and diisopropyl ether; ester solvents such as ethyl acetate and butyl acetate; aromatic solvents such as toluene and chlorobenzene; nitrile solvents such as acetonitrile and propionitrile; and mixed solvents thereof.

The amount of the solvent used is not limited. For example, it is practically 100 parts by weight or less based on 1 part by weight of 4-methylthio-2-oxo-1-butanal.

In Step A, the mixing order of the reaction reagents is not limited. A preferable embodiment is an embodiment in which, when Compound (2-1) is used, for example, 4-methylthio-2-oxo-1-butanal, Compound (2-1), the oxidizing agent, the alcohol, and the solvent if necessary, are mixed, and the base is added to the resulting mixture. When at least one compound selected from the group consisting of Compound (2-2) and Compound (2-3) is used, a preferable embodiment is, for example, a method in which 4-methylthio-2-oxo-1-butanal, the alcohol, at least one compound selected from the group consisting of Compound (2-2) and Compound (2-3), and the solvent if necessary, are mixed, and the oxidizing agent is added to the resulting mixture.

Step A is carried out in any condition of a reduced pressure, an ordinary pressure and an increased pressure, and is preferably carried out in an ordinary pressure or an increased pressure.

The reaction temperature in Step A varies depending on the kind or the amount of carbene catalyst used, and when using Compound (2-1), it varies also depending on the kind or the amount of the base used. The reaction temperature is preferably within a range of −20° C. to 150° C., more preferably within a range of 0° C. to 100° C. When the reaction temperature is lower than −20° C., the oxidation reaction speed tends to be lowered, and when the reaction temperature is higher than 150° C., the selectivity of the reaction tends to be reduced.

The degree of progress of the reaction in Step A can be confirmed by an analysis procedure such as gas chromatography, high-performance liquid chromatography, thin-layer chromatography, a nuclear magnetic resonance spectroscopic analysis, or an infrared absorption spectroscopic analysis.

After the reaction is finished in Step A, the oxidizing agent used is removed through degassing or filtration, then the obtained reaction mixture is subjected to, for example, a concentration treatment if necessary, followed by a cooling treatment or the like, whereby 4-methylthio-2-oxo-butyrate can be taken out.

The 4-methylthio-2-oxo-butyrate taken out can be purified through a purification procedure such as distillation, a column chromatography or crystallization.

Next, Step B of reductively aminating 4-methylthio-2-oxo-butyrate obtained in Step A will be explained. Methionine can be obtained by a reductive amination reaction of Step B.

In Step B, 4-methylthio-2-oxo-butyrate obtained in Step A is reductively aminated. Such a reductive amination reaction may be carried out using a reducing agent such as an alkali metal salt of aluminum hydride or a borohydride alkali metal salt, or may be carried out using hydrogen or formic acid as the reducing agent in the presence of a metal catalyst. The reductive amination reaction of Step B is preferably carried out by reacting 4-methylthio-2-oxo-butyrate, ammonia and the reducing agent in the presence of a transition metal.

The transition metal used in Step B (hereinafter sometimes referred to as a “transition metal catalyst”) is preferably at least one metal selected from the group consisting of noble metals, nickel, cobalt and copper, or preferably at least one metal selected from the group consisting of ruthenium, rhodium, palladium, platinum, iridium, nickel, cobalt and copper.

Examples of the noble metal include ruthenium, rhodium, palladium, platinum and iridium, and the noble metal is preferably supported on a carrier (hereinafter sometimes referred to as a “supported catalyst”). As the carrier, for example, at least one carrier selected from the group consisting of activated carbon, alumina, silica and zeolite is preferable.

Examples of the nickel include reduced nickel (hereinafter sometimes referred to as a “reduced nickel catalyst”) and sponge nickel (Raney® nickel) (hereinafter sometimes referred to as a “sponge nickel catalyst”); examples of the cobalt include reduced cobalt (hereinafter sometimes referred to as a “reduced cobalt catalyst”), sponge cobalt (Raney® cobalt) (hereinafter sometimes referred to as a “sponge cobalt catalyst”); and examples of the copper include sponge copper (Raney® copper) (hereinafter sometimes referred to as a “sponge copper catalyst”). The reduced metal catalyst such as the reduced nickel catalyst or the reduced cobalt catalyst is a catalyst produced by reducing a metal oxide or a metal hydroxide, or a catalyst produced by reducing a metal oxide supported on a carrier or a metal hydroxide supported on a carrier. In addition, the sponge metal catalyst such as the sponge nickel catalyst, the sponge cobalt catalyst or the sponge copper catalyst is a catalyst obtained by reacting an alloy of nickel and aluminum, an alloy of cobalt and aluminum or an alloy of copper and aluminum with an aqueous sodium hydroxide solution to dissolve aluminum.

The transition metal catalyst in Step B is preferably the sponge metal catalyst or a noble metal, and more preferably at least one catalyst selected from the group consisting of the sponge nickel catalyst, the sponge cobalt catalyst and the sponge copper catalyst, or a noble metal supported on a carrier. Of these, as the transition metal catalyst, palladium supported on activated carbon and rhodium supported on activated carbon are preferable.

The transition metal catalyst may be a commercially available product or may be produced by any publicly known method.

The amount of transition metal catalyst used is preferably 2 parts by weight or less in terms of transition metal atoms, more preferably within a range of 0.0001 to 0.2 part by weight, even more preferably within a range of 0.001 to 0.1 part by weight based on 1 part by weight of 4-methylthio-2-oxo-butyrate.

Ammonia used in Step B may be used in any state, for example, as liquid ammonia, an ammonia gas or an ammonia solution. An ammonia solution is preferably used, and aqueous ammonia and a methanol solution of ammonia are more preferably used. When aqueous ammonia is used, the concentration thereof is preferably 10 to 35% by weight. In addition, ammonia may be formed into a salt with, for example, an inorganic acid such as hydrochloric acid or sulfuric acid, or a carboxylic acid such as formic acid or acetic acid.

The amount of ammonia used is preferably 1 mole or more per mole of 4-methylthio-2-oxo-butyrate. The upper limit of ammonia used is not limited, and is, for example, 500 moles per mole of 4-methylthio-2-oxo-butyrate.

As the reducing agent used in Step B, it is preferable to use at least one substance selected from the group consisting of hydrogen and formic acid. As hydrogen, a commercially available hydrogen gas may be used, or hydrogen generated from, for example, formic acid or a salt thereof by any publicly known method may be used. When the hydrogen gas is used, its partial pressure is preferably 10 MPa or less, more preferably within a range of 0.01 to 5 MPa, further preferably within a range of 0.02 to 2 MPa, even more preferably within a range of 0.05 to 0.8 MPa. As the formic acid, commercially available formic acid may be used.

In Step B, the reductive amination reaction is preferably carried out in the presence of a solvent. Solvents which are inactive in the reductive amination reaction are preferable, and examples thereof include aliphatic hydrocarbon solvents such as pentane, hexane, isohexane, heptane, isoheptane, octane, isooctane, nonane, isononane, decane, isodecane, undecane, dodecane, cyclopentane, cyclohexane, methylcyclohexane, tert-butylcyclohexane and petroleum ethers; ether solvents such as tetrahydrofuran, methyl tetrahydrofuran, diethyl ether, dipropyl ether, diisopropyl ether, dibutyl ether, dipentyl ether, dihexyl ether, diheptyl ether, dioctyl ether, tert-butyl methyl ether, cyclopentyl methyl ether, 1,2-dimethoxyethane and diethyleneglycol dimethyl ether; alcohol solvents such as methanol, ethanol, 1-propanol, 2-propanol, 1-butanol, isobutyl alcohol, tert-butyl alcohol, 1-pentanol, 2-pentanol, isopentyl alcohol, 1-hexanol, 2-hexanol, isohexyl alcohol, 1-heptanol, 2-heptanol, 3-heptanol, isoheptyl alcohol, ethyleneglycol monomethyl ether, ethyleneglycol monoethyl ether, ethyleneglycol monopropyl ether, ethyleneglycol monoisopropyl ether, ethyleneglycol monobutyl ether, ethyleneglycol monoisobutyl ether, ethyleneglycol mono-tert-butyl ether, diethyleneglycol monomethyl ether, diethyleneglycol monoethyl ether, diethyleneglycol monopropyl ether, diethyleneglycol monoisopropyl ether, diethyleneglycol monobutyl ether, diethyleneglycol monoisobutyl ether and diethyleneglycol mono-tert-butyl ether; ester solvents such as ethyl acetate, propyl acetate, isopropyl acetate, butyl acetate, isobutyl acetated, tert-butyl acetate, amyl acetate and isoamyl acetate; aprotic polar solvents such as dimethyl sulfoxide, sulfolane, N,N-dimethyl formamide, N,N-dimethyl acetamide, N,N-dimethyl propionamide, N-methyl pyrrolidone, γ-butyrolactone, dimethyl carbonate, diethyl carbonate, ethylene carbonate, propylene carbonate, 1,3-dimethyl-2-imidazolidinone and 1,3-dimethyl-3,4,5,6-tetrahydro-2(1H)-pyridinone; water; and mixtures thereof. Of these, alcohol solvents and water are preferable, and methanol and water are more preferable. The amount of the solvent used is preferably within a range of 1 to 200 mL, more preferably within a range of 10 to 150 mL based on 1 gram of 4-methylthio-2-oxo-butyrate.

In Step B, the mixing order of reaction reagents is not limited. For example, there is a method in which 4-methylthio-2-oxo-butyrate, ammonia and the transition metal catalyst are mixed, and hydrogen is added to the resulting mixture, or a method in which 4-methylthio-2-oxo-butyrate and ammonium formate are mixed, to which formic acid is added if necessary to adjust the pH to a certain value, and the transition metal catalyst is added to the resulting mixture.

The reaction temperature in Step B is preferably within a range of 0° C. to 100° C., more preferably within a range of 20° C. to 90° C. The reaction time depends on the reaction temperature, the amount of the reaction reagent or solvent used, or the partial pressure of hydrogen, and is, for example, within a range of 1 to 24 hours. The degree of progress of the reaction of the reductive amination reaction can be confirmed by an analysis procedure such as thin-layer chromatography, gas chromatography or high-performance liquid chromatography.

After the reductive amination reaction is finished, the obtained reaction mixture is subjected to temperature adjustment if necessary, and then the resulting product is subjected to a post-treatment such as filtration, neutralization, extraction or washing with water, followed by an isolation treatment such as distillation, crystallization or solid-liquid separation, whereby methionine can be taken out. Specifically, for example, the temperature of the obtained reaction mixture is adjusted to around room temperature, or the reaction mixture is not subjected to the temperature adjustment, and then the transition metal catalyst is removed by filtration. After that, the obtained filtrate is neutralized to deposit methionine, and the deposited methionine can be recovered through filtration or the like. When the obtained reaction mixture exhibits basicity, for example, the neutralization is carried out by mixing the reaction mixture with an acid such as hydrochloric acid or carbonic acid, and when the reaction mixture exhibits acidity, it is carried out by mixing the reaction mixture with a base such as sodium carbonate, sodium bicarbonate or potassium carbonate. The transition metal catalyst removed through filtration or methionine recovered through filtration or the like can be washed with the solvent described above. In addition, the recovered methionine can be dried, for example, under reduced pressure. When the reaction mixture contains ammonia, ammonia can be removed from the reaction mixture by blowing a nitrogen gas into the reaction mixture.

The isolated methionine can be purified through a purification procedure such as recrystallization; extractive purification; distillation; an adsorption treatment to activated carbon, silica, alumina or the like; or chromatography such as silica-gel column chromatography.

### EXAMPLES

The present invention will be explained based on examples below.

Production example of 1,3-bis[(2,4,6-tribromo)phenyl]imidazolinium chloride, which is used as a raw material for producing a carbene catalyst (Reference Example 1)

Production example of 1,3-bis(2,4,6-tribromophenyl)imidazolinium chloride

A 300 mL flask, whose inside air had been replaced by nitrogen, was charged with 25 g of 2,4,6-tribromoaniline, 200 g of chloroform and 9.2 g of triethylamine. To the resulting mixture was added dropwise 11.5 g of oxalyl chloride at 0° C. over 30 minutes. The obtained mixture was stirred at 0° C. for 2 hours, then at room temperature for 18 hours. To the obtained reaction mixture was added 100 g of water to deposit crystals. After that, the deposited crystals were recovered through filtration, then the recovered material was washed with 10 g of water and 20 g of diethyl ether and dried to obtain 20.4 g of white crystals. It was confirmed by gas chromatography/mass spectrometry (GC-MS) that the obtained white crystals were N,N′-bis(2,4,6-tribromophenyl)ethanediamide. Yield: 76%. MS (m/z): 713 (M⁺)

A 200 mL stainless steel autoclave was charged with 10.1 g of N,N′-bis(2,4,6-tribromophenyl)ethanediamide obtained above and 85 mL of a 1 M solution of BH₃/tetrahydrofuran, and then the mixture was heated and stirred at 75° C. for 16 hours. After being cooled to room temperature, the reaction liquid was added to a mixed liquid of 170 g of methanol and 8.5 g of 35% hydrochloric acid little by little with stirring. A low-boiling substance was distilled away from the obtained reaction liquid, and 150 g of methanol was added to the residue. A low-boiling substance was distilled away again to obtain 9.1 g of white crystals. Yield: 89%.

It was confirmed by GC-MS that the obtained crystals were N,N′-bis(2,4,6-tribromophenyl)-1,2-ethanediamine hydrochloride.

MS (m/z): 685 (M⁺, free amine)

A 200 mL flask, whose inside air had been replaced by nitrogen, was charged with 9 g of N,N′-bis(2,4,6-tribromophenyl)-1,2-ethanediamine hydrochloride obtained above and 100 g of triethyl orthoformate. After the obtained mixture was refluxed for 1 hour, it was cooled to room temperature, thereby depositing crystals. Next, the deposited crystals were recovered through filtration, and then the recovered material was washed with 10 g of tetrahydrofuran and dried to obtain 3.1 g of white crystals. It was confirmed by ¹H-NMR that the obtained crystals were 1,3-bis[(2,4,6-tribromo)phenyl]imidazolinium chloride. Yield: 32%

¹H-NMR (6/ppm, DMSO-d6, tetramethylsilane standard): 4.66 (s, 4H), 8.3 (s, 4H), 9.70 (s, 1H)

### Production Example of methyl 4-(methylthio)-2-oxo-butyrate

### Step A

### Example 1

A 100 mL stainless steel pressure-resistant reaction tube equipped with a magnetic rotor was charged with 100 mg of 4-(methylthio)-2-oxo-1-butanal, 20 mg of 3-(2,6-diisopropyl)phenyl-4,5-dimethylthiazolium chloride, 500 mg of methanol and 3 g of tetrahydrofuran, and the obtained mixture was cooled in a dry ice bath having a temperature of −70° C. while a nitrogen gas was blown into the mixture. After 2 g of dry ice and 6 mg of sodium methylate were added to the cooled mixture, the pressure-resistant reaction tube was sealed. The obtained mixture was reacted by stirring at 60° C. for 4 hours.

After the reaction was finished, carbon dioxide and by-produced carbon monoxide were removed as gases from the reaction mixture. After that, the obtained reaction mixture was analyzed by gas chromatography according to an internal standard method, and it was found that the yield of methyl 4-(methylthio)-2-oxo-butyrate was 50%. In the reaction mixture after the reaction was finished, 10% of 4-(methylthio)-2-oxo-1-butanal remained unreacted.

### Production Example of methyl 4-(methylthio)-2-oxo-butyrate

### Step A

### Example 2

A 100 mL stainless steel pressure-resistant reaction tube equipped with a magnetic rotor was charged with 100 mg of 4-(methylthio)-2-oxo-1-butanal, 18 mg of 3-(2,6-diisopropyl)phenyl-4,5-dimethylthiazolium chloride, 300 mg of methanol and 3 g of tetrahydrofuran, and the obtained mixture was cooled in a dry ice bath having a temperature of −70° C. while a nitrogen gas was blown into the mixture. After 2 g of dry ice and 10 mg of a 28% methanol solution of sodium methylate were added to the cooled mixture, the pressure-resistant reaction tube was sealed. After the obtained mixture was pressurized to 1 MPa with air, it was reacted by stirring at 60° C. for 3 hours.

After the reaction was finished, the reaction mixture was cooled to room temperature, and then the pressure was returned to an ordinary pressure by pressure discharge. The obtained reaction mixture was analyzed by gas chromatography according to an internal standard method, and it was found that the yield of methyl 4-(methylthio)-2-oxo-butyrate was 20%. In the reaction mixture after the reaction was finished, 20% of 4-(methylthio)-2-oxo-1-butanal remained unreacted.

### Production Example of methyl 4-(methylthio)-2-oxo-butyrate

### Step A

### Example 3

The same procedure as in Example 2 was performed except that 36 mg of 1,3-bis[(2,4,6-tribromo)phenyl]imidazolinium chloride was used instead of mg of 3-(2,6-diisopropyl)phenyl-4,5-dimethylthiazolium chloride in <Step A—Example 2>. Methyl 4-(methylthio)-2-oxo-butyrate was analyzed and it was found that its yield was 57%. In the reaction mixture after the reaction was finished, 6% of 4-(methylthio)-2-oxo-1-butanal remained unreacted.

### Production Example of methyl 4-(methylthio)-2-oxo-butyrate

### Step A

### Example 4

A 100 mL Schlenk tube equipped with a magnetic rotor was charged with 100 mg of 4-(methylthio)-2-oxo-1-butanal, mg of 1,3-bis[(2,4,6-tribromo)phenyl]imidazolinium chloride, 300 mg of methanol and 3 g of tetrahydrofuran, to which 10 mg of a 28% methanol solution of sodium methylate was added, and the mixture was reacted by stirring in an air atmosphere at 60° C. for 3 hours.

After the reaction was finished, the reaction mixture was cooled to room temperature. After that, the obtained reaction mixture was analyzed by gas chromatography according to an internal standard method, and it was found that the yield of methyl 4-(methylthio)-2-oxo-butyrate was 65%. In the reaction mixture after the reaction was finished, 4% of 4-(methylthio)-2-oxo-1-butanal remained unreacted.

### Production Example of methyl 4-(methylthio)-2-oxo-butyrate

### Step A

### Example 5

A 100 mL Schlenk tube equipped with a magnetic rotor was charged with 1 g of 4-(methylthio)-2-oxo-1-butanal, 300 mg of 1,3-bis[(2,4,6-tribromo)phenyl]imidazolinium chloride,
3 g of methanol and 10 g of tetrahydrofuran, to which 70 mg of a 28% methanol solution of sodium methylate was added, and the mixture was reacted by stirring in an air atmosphere at 60° C. for 3 hours.

After the reaction was finished, the reaction mixture was cooled to room temperature. After that, the obtained reaction mixture was analyzed by gas chromatography according to an internal standard method, and it was found that the yield of methyl 4-(methylthio)-2-oxo-butyrate was 30%. In the reaction mixture after the reaction was finished, 40% of 4-(methylthio)-2-oxo-1-butanal remained unreacted.

### Production Example of methyl 4-(methylthio)-2-oxo-butyrate

### Step A

### Example 6

A 100 mL stainless steel pressure-resistant reaction tube equipped with a magnetic rotor was charged with 100 mg of 4-(methylthio)-2-oxo-1-butanal, 20 mg of 2-methoxy-1,3-bis[(2,6-diisopropyl)phenyl]imidazolidine, 500 mg of methanol and 2 g of tetrahydrofuran in a nitrogen atmosphere, and the obtained mixture was cooled in a dry ice bath having a temperature of −70° C. After 2 g of dry ice was added to the cooled mixture, the pressure-resistant reaction tube was sealed. The obtained mixture was reacted by stirring at 60° C. for 6 hours.

After the reaction was finished, carbon dioxide and by-produced carbon monoxide were removed as gases from the reaction mixture. After that, the obtained reaction mixture was analyzed by gas chromatography according to an internal standard method, and it was found that the yield of methyl 4-(methylthio)-2-oxo-butyrate was 20%. In the reaction mixture after the reaction was finished, 30% of 4-(methylthio)-2-oxo-1-butanal remained unreacted.

### Production Example of methyl 4-(methylthio)-2-oxo-butyrate

### Step A

### Example 7

A 100 mL stainless steel pressure-resistant reaction tube equipped with a magnetic rotor was charged with 100 mg of 4-(methylthio)-2-oxo-1-butanal, 10 mg of 2-carboxy-4,5-dihydro-1,3-bis[(2,4,6-trimethyl)phenyl]imidazolium, 500 mg of methanol and 3 g of tetrahydrofuran in a nitrogen atmosphere, and the obtained mixture was cooled in a dry ice bath having a temperature of −70° C. After 2 g of dry ice was added to the cooled mixture, the pressure-resistant reaction tube was sealed. The obtained mixture was reacted by stirring at 60° C. for 4 hours.

After the reaction was finished, carbon dioxide and by-produced carbon monoxide were removed as gases from the reaction mixture. After that, the obtained reaction mixture was analyzed by gas chromatography according to an internal standard method, and it was found that the yield of methyl 4-(methylthio)-2-oxo-butyrate was 10%.

Analysis Method in Step B

In Step B—Example 1 described below, a reaction mixture was analyzed using high-performance liquid chromatography (manufactured by Shimadzu Corporation) under analysis conditions described below, and a conversion and a selectivity were calculated in accordance with the following equations.

### <Analysis Conditions>

| LC Column | Lichrosorb-RP-8 | |
|---|---|---|
| Column | 40° C. | |
| Temperature | | |
| Mobile Phase | acetonitrile/water = | 5/95 |
| | Additive | sodium 1- |
| | | pentanesulfonate |
| | Concentration of | 2.5 mmol/L |
| | Additive | |
| | pH of Mobile Phase | 3 (adjusted by adding |
| | | 40% phosphoric acid) |
| Flow Rate | 1.5 mL/minute | |
| Detection | 210 nm | |
| Wavelength | | |
| Measurement | 60 minutes | |
| Time | | |

### <Calculation of Conversion>

Conversion(%)=100(%)−(peak area of methyl 4−(methylthio)-2-oxo-butyrate(%))<

### <Calculation of Selectivity>

Selectivity(%)=(peak area of methionine)/(peak areas of all products)×100

### Production Example of Methionine

### Step B

### Example 1

An autoclave having an inner capacity of 60 mL was charged with 130 mg of methyl 4-(methylthio)-2-oxo-butyrate, 5.4 g of 28% aqueous ammonia and 80 mg of 5% Pd/C (manufactured by N.E. CHEMCAT Corporation), and the obtained mixture was stirred. After hydrogen was pressed into the autoclave to adjust the inner pressure to 0.5 MPaG (gauge pressure), the temperature was elevated to 50° C., and the mixture was stirred at 50° C. for 6 hours. Part of the obtained reaction mixture was analyzed by high-performance liquid chromatography, and it was found that the conversion of methyl 4-(methylthio)-2-oxo-butyrate was 94.5% and the selectivity of methionine was 18%.

The present invention is industrially applicable as a production method of methionine.

1. A method for producing methionine comprising:
Step A of oxidizing 4-methylthio-2-oxo-1-butanal in the presence of an alcohol; and
Step B of reductively aminating 4-methylthio-2-oxo-butyrate obtained in Step A.

2. The production method according to claim 1, wherein Step A is carried out by reacting 4-methylthio-2-oxo-1-butanal, the alcohol and an oxidizing agent in the presence of a carbene catalyst.

3. The production method according to claim 2, wherein the carbene catalyst in Step A is at least one compound selected from the group consisting of a compound obtained by reacting a compound represented by the formula (2-1): 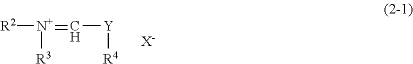
wherein R² is an optionally substituted alkyl group or an optionally substituted aryl group, R³ and R⁴ are each independently an optionally substituted alkyl group or an optionally substituted aryl group, or R³ and R⁴ are combined together to form an optionally substituted bivalent hydrocarbon group or an optionally substituted group represented by —CH═N—, Y is a group represented by —S— or a group represented by —N(R⁵)—, R⁵ is an optionally substituted alkyl group or an optionally substituted aryl group, or R⁵ and R⁴ are combined together to form an optionally substituted bivalent hydrocarbon group, and X⁻ is an anion, with a base; a compound represented by the formula (2-2): 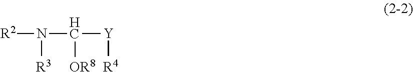
wherein R², R³, R⁴ and Y are each as defined above, and R⁸ is an alkyl group; a compound obtained by decomposing the compound represented by the formula (2-2); a compound represented by the formula (2-3): 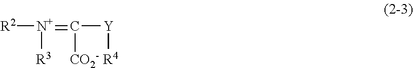
wherein R², R³, R⁴ and Y are each as defined above; and a compound obtained by decomposing the compound represented by the formula (2-3).

4. The production method according to claim 2, wherein the oxidizing agent in Step A is oxygen and/or carbon dioxide.

5. The production method according to claim 1, wherein the alcohol is methanol or ethanol.

6. The production method according to claim 1, wherein Step B is carried out in the presence of a solvent.

7. The production method according to claim 6, wherein the solvent in Step B is methanol or water.

8. The production method according to claim 1, wherein Step B is carried out by reacting 4-methylthio-2-oxo-butyrate, ammonia and a reducing agent in the presence of a transition metal.

9. The production method according to claim 8, wherein the transition metal in Step B is at least one metal selected from the group consisting of ruthenium, rhodium, palladium, platinum, iridium, nickel, cobalt and copper.
